(19) United States
(12) Patent Application Publication
(10) Pub. No.: US 2024/0057879 A1
(43) Pub. Date: Feb. 22, 2024

(54) BLOOD PRESSURE MEASUREMENT SYSTEM AND METHOD OF THE SAME

(71) Applicant: SAMSUNG DISPLAY CO., LTD., YONGIN-SI (KR)

(72) Inventor: Chul KIM, YONGIN-SI (KR)

(21) Appl. No.: 18/192,354

(22) Filed: Mar. 29, 2023

(30) Foreign Application Priority Data
Aug. 18, 2022 (KR) 10-2022-0103138

(51) Int. Cl. A61B 5/021; A61B 5/022

(52) U.S. Cl. CPC A61B 5/02116; A61B 5/02225

(57) ABSTRACT

A blood pressure measurement system includes a display device; and a pressure measurement device applying a pressure to a user's body according to a pressure control signal and sensing a pressure measurement value. The display device includes a pixel emitting light; a photo-sensor sensing external light and outputting a first pulse wave signal based on the external light; and a main processor, wherein the main processor receives the first pulse wave signal, outputs the pressure control signal to the pressure measurement device, receives a first pressure signal including the pressure measurement value from the pressure measurement device, and calculates a blood pressure based on the first pulse wave signal.

### CROSS-REFERENCE TO RELATED APPLICATION

This application claims priority under 35 U.S.C. 119 to Korean Patent Application No. 10-2022-0103138, filed on Aug. 18, 2022, in the Korean Intellectual Property Office, the disclosure of which is incorporated by reference in its entirety.

### TECHNICAL FIELD

The present disclosure relates to a blood pressure measurement system and a method of the same.

### DESCRIPTION OF THE RELATED ART

Display devices are devices that display visual information on screens. Display devices have been used in various applications such as televisions (TVs), monitors, mobile smartphones, tablets, and personal computers (PCs). Portable display devices are provided with various functions such as a camera function and a fingerprint sensor function.

Recently, as the healthcare industry has attracted attention, there have been efforts to develop methods for more conveniently acquiring biometric information about health. For example, there have been efforts to replace a traditional oscillometric pulse measurement device with an electronic product that is portable and convenient to carry. However, an electronic pulse measurement device requires an independent light source, sensor, and display included in the device, which makes the electronic pulse measurement device less convenient to carry.

### SUMMARY

Aspects of the present disclosure provide a blood pressure measurement system and a method of the same capable of measuring a blood pressure of a user by sensing a pressure applied by a user by a pressure measurement device, sensing light reflected from a finger blood vessel or the like of the user by a photo-sensor of a display panel, and analyzing a pulse wave signal according to an amount of sensed light and a pressure signal. Aspects of the present disclosure also provide a blood pressure measurement system and a method of the same capable of improving accuracy of blood pressure calculation by sensing an accurate pressure signal.

However, aspects of the present disclosure are not restricted to those set forth herein. The above and other aspects of the present disclosure will become more apparent to one of ordinary skill in the art to which the present disclosure pertains by referencing the detailed description of the present disclosure given below.

According to an embodiment of the disclosure, a blood pressure measurement system includes a display device and a pressure measurement device applying a pressure to a user's body according to a pressure control signal and sensing a pressure measurement value. The display device includes a pixel emitting light; a photo-sensor sensing external light and outputting a first pulse wave signal based on the external light; and a main processor, wherein the main processor receives the first pulse wave signal, outputs the pressure control signal to the pressure measurement device, receives a first pressure signal including the pressure measurement value from the pressure measurement device, and calculates a blood pressure based on the first pulse wave signal and the first pressure signal.

In an embodiment, the pressure measurement device includes a pressure applying part applying the pressure to the user's body, a pressure sensor sensing an external pressure, and a controller controlling the pressure applying part.

In an embodiment, the main processor controls the pressure applying part so that the pressure measurement value gradually increases over time.

In an embodiment, the pressure measurement device further includes a wireless communication module receiving the pressure control signal.

In an embodiment, when the pressure sensor senses the pressure of the user's body, the photo-sensor senses light reflected from the same user's body.

In an embodiment, the main processor receives the first pressure signal and calculates a non-measurement section in which the pressure measurement value is constant over time and a measurement section in which the pressure measurement value changes over time.

In an embodiment, the main processor extracts the first pressure signal in a section corresponding to the measurement section to calculate a second pressure signal, and extracts the first pulse wave signal in the section corresponding to the measurement section to calculate a second pulse wave signal.

In an embodiment, the main processor further calculates a third pulse wave signal including a pulse wave signal value according to a pressure based on the second pulse wave signal and the second pressure signal.

In an embodiment, the main processor generates a peak detection signal based on an amplitude corresponding to a peak of each of cycles of the third pulse wave signal.

In an embodiment, the main processor calculates a peak value of the peak detection signal and a pressure value corresponding to the peak value of the peak detection signal, and calculates a diastolic blood pressure lower than the pressure value, a systolic blood pressure higher than the pressure value, and a mean blood pressure according to the pressure value.

In an embodiment, the main processor calculates the mean blood pressure as the pressure value corresponding to the peak value.

In an embodiment, the main processor calculates a first pressure value smaller than the pressure value corresponding to 60% to 80% of the peak value in the peak detection signal and a second pressure value greater than the pressure value, and calculates the first pressure value as the diastolic blood pressure and calculates the second pressure value as the systolic blood pressure.

In an embodiment, one cycle of the third pulse wave signal includes a plurality of waveforms having different amplitudes, and when a peak value of a first waveform of the plurality of waveforms is defined as a pulse wave contraction value, a peak value of a second waveform of the plurality of waveforms is defined as a reflected pulse wave value, the pulse wave contraction value is defined as Sp, the reflected pulse wave value is defined as Rp, and a reflected pulse wave ratio is defined as RI, the main processor calculates the reflected pulse wave ratio by RI=Rp/Sp.

In an embodiment, the reflected pulse wave ratio includes a first period in which the reflected pulse wave ratio fluctuates within a first range, a second period in which the reflected pulse wave ratio fluctuates within a second range, and a third period in which the reflected pulse wave ratio fluctuates within a third range, and a width of the first range and a width of the third range are smaller than a width of the second range.

In an embodiment, the main processor analyzes the reflected pulse wave ratio to detect a start point in time of the second period, calculates a third pressure value corresponding to the first pulse wave signal at the start point in time of the second period and sets the third pressure value as a diastolic blood pressure, and calculates a fourth pressure value corresponding to the first pulse wave signal at a start point in time of the third period after the second period is calculated and sets the fourth pressure value as a systolic blood pressure.

According to an embodiment of the disclosure, a blood pressure measurement method of a blood pressure measurement system including a pressure measurement device applying a pressure to a user's body and sensing a pressure measurement value; and a display device including a pixel emitting light and a photo-sensor sensing external light and outputting a first pulse wave signal based on the external light, the blood pressure measurement method including applying a pressure control signal to the pressure measurement device for controlling the pressure measurement device; receiving a first pressure signal including the pressure measurement value during a period of time from the pressure measurement device; receiving the first pulse wave signal from the photo-sensor; calculating, based on the first pressure signal, a non-measurement section and a measurement section for a period of time, wherein during the non-measurement section the pressure measurement value is constant and during the measurement section the pressure measurement value varies; extracting the first pressure signal in a section corresponding to the measurement section to calculate a second pressure signal and extracting the first pulse wave signal in the section corresponding to the measurement section to calculate a second pulse wave signal; and calculating a blood pressure based on the second pulse wave signal and the second pressure signal.

In an embodiment, the first pressure signal gradually increases over time in the measurement section.

In an embodiment, further comprising calculating a third pulse wave signal including a pulse wave signal value according to a pressure based on the second pulse wave signal and the second pressure signal and generating a peak detection signal based on an amplitude corresponding to a peak of each of cycles of the third pulse wave signal.

In an embodiment, further comprising calculating a peak value of the peak detection signal and a pressure value corresponding to the peak value of the peak detection signal and calculating a diastolic blood pressure lower than the pressure value, a systolic blood pressure higher than the pressure value, and a mean blood pressure according to the pressure value.

In an embodiment, the mean blood pressure is calculated as the pressure value corresponding to the peak value

In a case of the blood pressure measurement method of the blood pressure measurement system according to the present embodiment, the pressure measurement device may measure the pressure applied to the artery of the user, and the display device 1 may measure the pulse wave signal. The blood pressure measurement system according to the present embodiment may more accurately measure the pressure by applying the pressure to the user's body and measuring the pressure applied to the user's body.

### BRIEF DESCRIPTION OF THE DRAWINGS

The above and other aspects and features of the present disclosure will become more apparent by describing in detail embodiments thereof with reference to the attached drawings, in which:

FIG. 1 is a perspective view illustrating a blood pressure measurement system according to an embodiment;

FIG. 2 is a block diagram illustrating the blood pressure measurement system according to an embodiment;

FIG. 3 is a plan view illustrating the blood pressure measurement system according to an embodiment;

FIG. 4 is a flowchart illustrating a pressure measurement method of a pressure measurement device according to an embodiment;

FIG. 5 is a graph illustrating a first pressure signal calculated by the pressure measurement device;

FIG. 6 is a plan view illustrating a display device according to an embodiment;

FIG. 7 is a block diagram illustrating the display device according to an embodiment;

FIG. 8 is a plan layout view of pixels and photo-sensors of a display panel according to an embodiment;

FIG. 9 is a circuit diagram illustrating a pixel and a photo-sensor according to an embodiment in detail;

FIG. 10 is a block diagram illustrating a main processor according to an embodiment;

FIG. 11 is a flowchart illustrating a blood pressure calculation method of the blood pressure measurement system according to an embodiment;

FIG. 12 is a graph illustrating a second pressure signal during a period of time;

FIGS. 13 and 14 are graphs illustrating pulse wave signals during a period of time;

FIG. 15 is a graph illustrating a pulse wave signal according to a pressure;

FIG. 16 is a flowchart illustrating a method of calculating a blood pressure according to an embodiment;

FIG. 17 is a graph illustrating a waveform of a peak detection signal;

FIG. 18 is a flowchart illustrating a method of calculating a blood pressure according to another embodiment;

FIG. 19 is a graph illustrating a waveform of one cycle of a pulse wave signal according to another embodiment; and

FIG. 20 is a graph illustrating a pulse wave signal and a reflected pulse wave ratio according to another embodiment.

### DETAILED DESCRIPTION

The present invention will now be described in detail hereinafter with reference to the accompanying drawings, in which embodiments of the invention are shown. This invention may, however, be embodied in different forms and should not be construed as limited to the embodiments set forth herein.

It will also be understood that when a layer is referred to as being “on” another layer or substrate, it can be directly on the other layer or substrate, or intervening layers may also be present. The same reference numbers indicate the same components throughout the specification.

It will be understood that, although the terms “first,” “second,” etc. may be used herein to describe various elements, these elements should not be limited by these terms. These terms are only used to distinguish one element from another element. For instance, a first element discussed below could be termed a second element without departing from the teachings of the present invention. Similarly, the second element could also be termed the first element.

Each of the features of the various embodiments of the present disclosure may be combined or combined with each other, in part or in whole, and technically various interlocking and driving are possible. Each embodiment may be implemented independently of each other or may be implemented together in an association.

Hereinafter, embodiments of the present disclosure will be described with reference to the accompanying drawings.

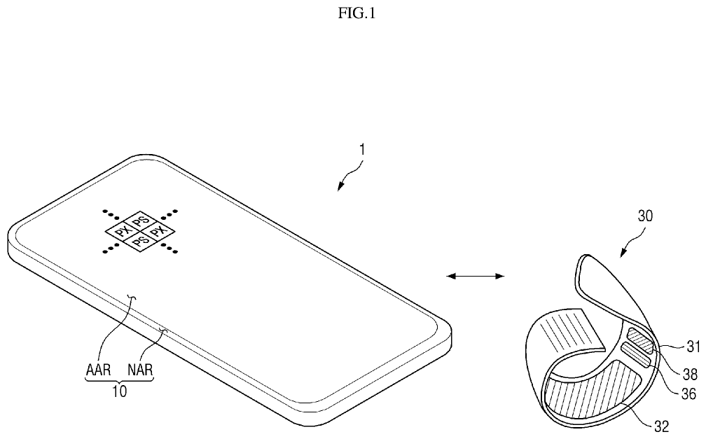

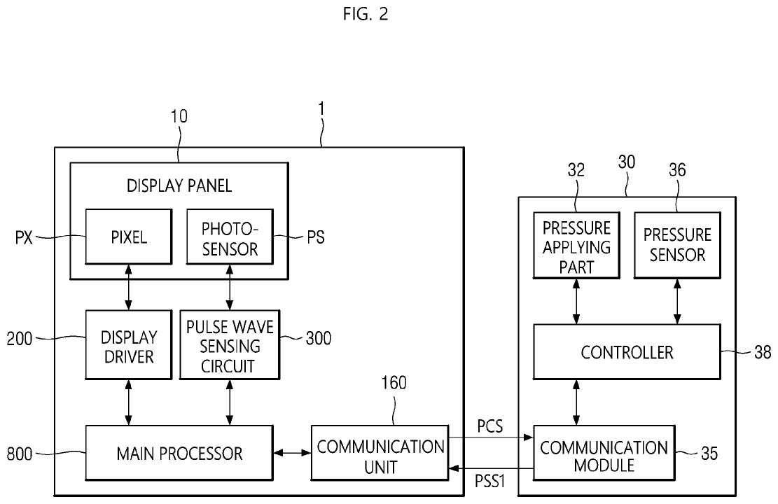

FIG. 1 is a perspective view illustrating a blood pressure measurement system according to an embodiment. FIG. 2 is a block diagram illustrating the blood pressure measurement system according to an embodiment.

In FIG. 2, a first direction X, a second direction Y, and a third direction Z are indicated. The first direction X is a direction parallel to one side of a display device 1 in a plan view, and may represent, for example, a transverse direction of the display device 1. The second direction Y is a direction parallel to the other side of the display device 1 in contact with one side of the display device 1 in a plan view, and may represent a longitudinal direction of the display device 1. Hereinafter, for convenience of explanation, one side in the first direction X refers to a right direction in a plan view, the other side in the first direction X refers to a left direction in a plan view, one side in the second direction Y refers to an upper direction in a plan view, and the other side in the second direction Y refers to a lower direction in a plan view. A third direction Z may represent a thickness direction of the display device 1. However, it is to be understood that directions mentioned in embodiments refer to relative directions, and embodiments might not be necessarily limited to the mentioned directions.

Referring to FIGS. 1 and 2, a blood pressure measurement system according to an embodiment includes a display device 1 and a pressure measurement device 30. The display device 1 may include a display panel 10, a display driver 200, a pulse wave sensing circuit 300, a main processor 800, and a communication unit 160.

The display device 1 may include various electronic devices that provide display screens. Examples of the display device 1 may include, but might not be necessarily limited to, mobile phones, smartphones, tablet personal computers (PCs), mobile communication terminals, electronic notebooks, electronic books, personal digital assistants (PDAs), portable multimedia players (PMPs), navigation devices, ultra mobile PCs (UMPCs), televisions, game machines, wrist watch-type electronic devices, head-mounted displays, monitors of personal computers, laptop computers, vehicle instrument boards, digital cameras, camcorders, external billboards, electric signs, various medical devices, various inspection devices, various home appliances including display areas, such as refrigerators and washing machines, Internet of Things (IoT) devices, or the like. Representative examples of a display device 1 to be described later may include smartphones, tablet PCs, laptop computers, or the like, but are not limited thereto.

The display device 1 includes the display panel 10 having an active area AAR and a non-active area NAR.

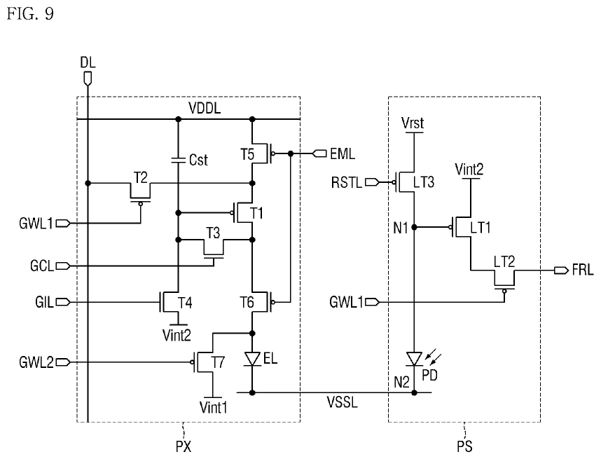

The active area AAR includes a display area in which a screen is displayed. The active area AAR may completely overlap the display area. A plurality of pixels PX displaying an image may be disposed in the display area. Each pixel PX may include a light emitting element EL (see FIG. 9).

The active area AAR further includes a light sensing area. The light sensing area is an area sensing light. For example, the light sensing area may be configured to sense an amount of external light of a wavelength, or the like. For example, the light sensing area may sense an incident light. The light sensing area may overlap the display area. In an example embodiment, the light sensing area may be defined as an area the same as the display area in a plan view. In another example, the light sensing area may be disposed only in a limited area required for blood pressure measurement. In this case, the light sensing area may overlap a portion of the display area, but may not overlap another portion of the display area.

A plurality of photo-sensors PS responding to light may be disposed in the active area AAR. Each photo-sensor PS may include a photoelectric conversion element PD (see FIG. 9) sensing incident light and converting the incident light into an electrical signal.

The non-active area NAR is disposed around the active area AAR. The non-active area NAR may be a bezel area. The non-active area NAR may surround all sides, for example, four sides, (as illustrated in FIG. 1) of the active area AAR, but might not be necessarily limited thereto.

Signal lines or driving circuits for applying signals to the active area AR may be disposed in the non-active area NAR. In addition, signal lines or driving circuits for applying signals to the light sensing area and light sensing lines for transferring electrical signals transferred from the light sensing area may be disposed in the non-active area NAR. In some cases, the non-active area NAR may not include the display area. In some cases, the non-active area NAR may not include the light sensing area. In another embodiment, the non-active area NAR may include a portion of the light sensing area. The non-active area NAR may be the same area as the non-display area.

The display driver 200 may drive the plurality of pixels PX and/or the plurality of photo-sensors PS. The display driver 200 may output signals and voltages for driving the display panel 10. The display driver 200 may be formed as an integrated circuit (IC) and be mounted on the display panel 10. Signal lines for transferring signals between the display driver 200 and the active area AAR may be disposed in the non-active area NAR.

The pulse wave sensing circuit 300 may be connected to a display layer of the display panel 10. The pulse wave sensing circuit 300 may sense a photocurrent, for example, the photocurrent generated by photo charges incident on the plurality of photo-sensors PS of the display panel 10. The pulse wave sensing circuit 300 may identify a pulse wave of a user based on the photocurrent.

The main processor 800 may control all functions of the display device 1. For example, the main processor 800 may output digital video data to the display driver 200 through a circuit board 311 so that the display panel 10 displays an image. In addition, the main processor 800 may receive touch data from a touch driving circuit, determine touch coordinates of the user on the display device, and execute an application indicated by an icon displayed at the user-specified touch coordinates.

The main processor 800 may calculate a pulse wave signal PPG reflecting a change of blood pressure depending on a heartbeat according to an optical signal input from the pulse wave sensing circuit 300. In addition, the main processor 800 may calculate a pressure measurement value of the user according to a first pressure signal PSS1 input from the pressure measurement device 30. In addition, the main processor 800 may calculate a blood pressure of the user based on the pulse wave signal PPG and a pressure signal.

The communication unit 160 may perform wired/wireless communication with an external device. For example, the communication unit 160 may transmit and receive communication signals to and from a communication module 35 of the pressure measurement device 30. The communication unit 160 may receive a pressure control signal PCS from the main processor 800 and provide the pressure control signal PCS to the pressure measurement device 30. In addition, the communication unit 160 may receive the first pressure signal PSS1 from the pressure measurement device 30 and may provide the first pressure signal PSS1 to the main processor 800. Examples of devices that may perform the functions described in the communication unit 160 include modems and transceivers. A modem is a device that modulates an analog carrier signal to encode digital information, and demodulates such a carrier signal to decode the transmitted information. For example, a modem in the communication unit 160 can be used to transmit and receive digital information between the device and the pressure measurement device 30 through wired or wireless communication. A transceiver is a device that includes both a transmitter and a receiver. For example, a transceiver in the communication unit 160 can be used to both transmit and receive communication signals, pressure control signals and pressure measurement signals to and from the main processor 800 and the pressure measurement device 30 through wired or wireless communication These modems and the transceivers can perform wired or wireless communication with external devices, such as a communication module 35 of a pressure measurement device 30. The modems and the transceivers can transmit and receive communication signals, provide pressure control signals and pressure measurement signals to and from the main processor 800 and the pressure measurement device 30.

A pressure applying part 32 according to an embodiment may apply a pressure to a user's body and measure the pressure applied to the user's body. The pressure applying part 32 may accurately determine the pressure applied to the user's body during a period of time by constantly applying the pressure to the user's body. In some cases, the pressure measurement device 30 may operate independently, without being attached to or connected to an external device.

The pressure measurement device 30 includes the pressure applying part 32, a pressure sensor 36, the communication module 35, and a controller 38. The pressure applying part 32 is configured to accommodate a user's finger. One example of the controller 38 is a control circuit. A control circuit typically includes a combination of electronic components such as switches, sensors, actuators, and processors that are interconnected to perform specific tasks. The control circuit receives input from sensors, processes the input, and generates control signals to actuate other devices or systems. A control circuit may be responsible for controlling the operation of the pressure measurement device 30. A control circuit may be used for managing the communication between the pressure sensor 36 and the communication module 35, and coordinating the operation of the pressure applying part 32, which is designed to accommodate a user's finger, with the pressure sensor 36. The control circuit may also be used for controlling sending and receiving data through the communication module 35, such as pressure measurements and control signals, to the main processor 800 or other external devices.

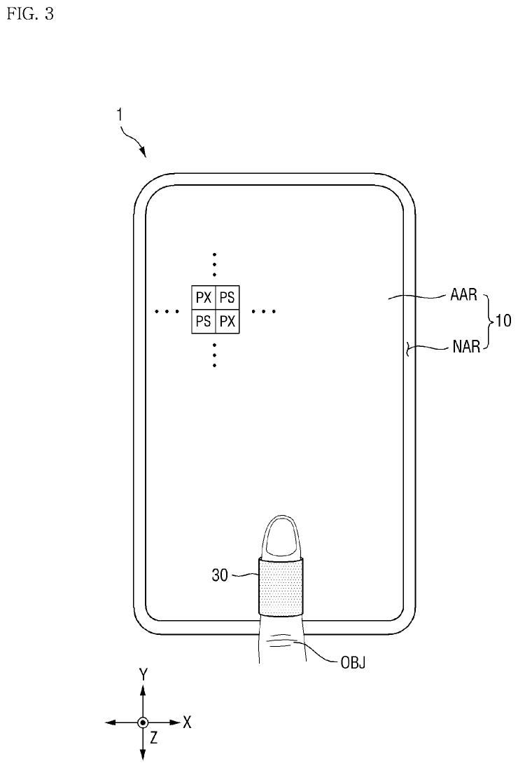

The pressure applying part 32 directly applies a pressure to the user's body, for example, an artery. The pressure applying part 32 operates to occlude a blood flow in the artery of the finger by gradually applying the pressure. For example, the pressure applying part 32 may be a finger cuff disposed to be positioned on the user's finger, as illustrated in FIG. 3. The pressure sensor 36 is disposed in a position, for example adjacent to the pressure applying part 32, to sense a pressure measurement value from a pressure application site such as the finger. The controller 38 receives and processes a sensor signal (e.g., a pressure measurement value during a period of time) from the pressure sensor 36 to generate a pressure signal.

The pressure applying part 32 may be implemented using an air inflation mechanism or a pneumatic system that inflates air into the pressure applying part 32. In one example, a pressure applying element is an inflatable finger cuff. The finger cuff may include an inflation mechanism or a pneumatic system that inflates air into the inflatable cuff. Alternatively, in another embodiment, the pressure applying element may be implemented using a compression mechanism that applies a force directly to the artery in order to occlude a blood flow in the artery. In one example, the pressure measurement device 30 may be implemented as a mechanical spring.

A support part 31 may be configured as a wrap around cuff. In some cases, the support part 31 includes a pressure applying part 32 formed as an open cuff. One end of the pressure applying part 32 may be closed using any re-attachable means such as Velcro. However, the present disclosure might not be necessarily limited thereto, and the pressure applying part 32 may be formed integrally with the support part 31. In this case, the pressure applying part 32 may be an air injection-type device disposed in the support part 31 and injecting air into the finger cuff. Alternatively, in another example, the support part 31 may have a ring or semi-ring shape of which a size is adjustable.

The pressure sensor 36 may be attached to one surface of the support part 31. The pressure sensor 36 may sense an electrical signal due to the pressure applied to the user's body. The pressure sensor 36 may generate pressure data according to a change in the electrical signal sensed by the pressure sensor, and transmit the pressure data to the controller 38.

The controller 38 operates the pressure measurement device 30 so that the pressure applying part 32 may expand and contract, and controls the pressure sensor 36 to collect and store sensed signals. The controller 38 implements the blood pressure measurement system according to the present disclosure in order to provide pressure measurement values based on the sensed signal. In some embodiments, the controller 38 controls the communication module 35 so that sensed pressure measurement values and/or sensed signal data are communicated for example, wirelessly, with other devices. Accordingly, the controller 38 receives the pressure measurement value applied to the user's body, and calculates the first pressure signal PSS1 based on the pressure measurement value. The first pressure signal PSS1 may be provided to the display device 1 using the communication module 35.

The communication module 35 may perform wired/wireless communication with an external device. For example, the communication module 35 may transmit and receive communication signals to and from the communication unit 160 of the display device 1. The communication module 35 may receive the first pressure signal PSS1 from the controller 38, and may provide the first pressure signal PSS1 to the display device 1. The communication module 35 may receive the pressure control signal PCS from the display device 1 and provide the pressure control signal PCS to the controller 38.

However, the present disclosure might not be necessarily limited thereto, and in another embodiment, the pressure measurement device 30 is configured as an additional device for an electronic device such as a mobile device or a wearable device. In this case, the pressure measurement device 30 may be connected to an external electronic device such as a user-wearable device that transfers data and/or receives a control signal.

FIG. 3 is a plan view illustrating the blood pressure measurement system according to an embodiment.

Referring to FIG. 3, the blood pressure measurement system according to an embodiment includes the display device 1 and the pressure measurement device 30. The blood pressure measurement system may calculate a systolic/diastolic blood pressure.

The pressure measurement device 30 according to an embodiment is attached to a user's finger OBJ. The pressure measurement device 30 may measure a pressure applied to the user's finger OBJ. The pressure measurement device 30 applies a gradually increasing pressure to the artery of the user OBJ until the pressure reaches a specific pressure level so that the artery is completely occluded and blood does not flow through the artery.

When the gradually increasing pressure is applied to the artery of the user OBJ, the display device 1 senses a pulse wave signal. For example, the user's finger OBJ may be in contact with the display panel 10 while a volume of the pressure applying part 32 increases, and the photo-sensor PS may sense the pulse wave signal reflected from the artery of the user OBJ. The pulse wave signal fluctuates according to a heartbeat cycle, and may thus reflect a change in blood pressure according to a heartbeat.

In this case, a body position of the user OBJ to which the pressure measurement device 30 is attached and a body position of the user OBJ in contact with the display device 1 may be adjacent to each other. Accordingly, the display device 1 may calculate a blood pressure of the user OBJ based on the pressure signal and the pulse wave signal sensed by the photo-sensor PS. According to some embodiments, the controller 38 controls the pressure sensor 36 and the photo-sensor PS to be synchronized and to simultaneously sense the external pressure from the user's body and light reflected from the user's body, respectively.

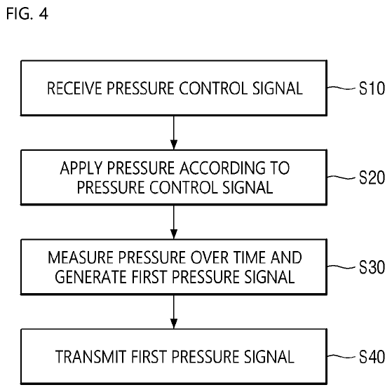

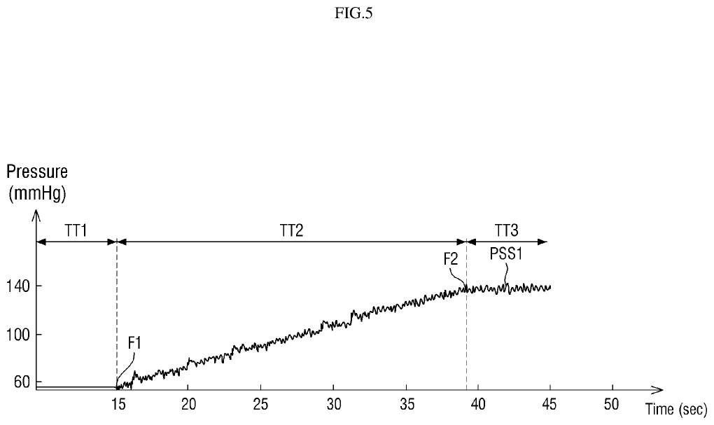

FIG. 4 is a flowchart illustrating a pressure measurement method of a pressure measurement device according to an embodiment. FIG. 5 is a graph illustrating a first pressure signal calculated by the pressure measurement device. A pressure measurement method of the pressure measurement device 30 will be described with reference to FIGS. 4 and 5.

Referring to FIG. 4, first, the controller 38 receives the pressure control signal PCS (S10).

The controller 38 may receive the pressure control signal PCS from the display device 1. For example, the communication module 35 of the pressure measurement device may receive the pressure control signal PCS from the communication unit 160 of the display device 1. The controller 38 may receive the pressure control signal PCS from the communication module 35. In this case, the pressure control signal PCS may be a signal for controlling the pressure measurement device 30 to apply a constantly increasing pressure to the user's body.

Next, the controller 38 controls the pressure applying part 32 according to the pressure control signal PCS, and accordingly, the pressure applying part 32 applies a pressure to the user's body (S20). The controller 38 may control the pressure applying part 32 according to the pressure control signal PCS. For example, when the pressure applying part 32 is implemented as the air inflation mechanism, the controller 38 may perform control to continuously apply air to the pressure applying part 32.

Accordingly, the pressure applying part 32 applies a pressure to the user's body. The pressure applying part 32 may apply a constantly increasing pressure to the user's body. That is, the pressure applying part 32 may be driven so that the user's body has a pressure that gradually increases during a period of time. For example, a volume of the pressure applying part 32 may gradually increase during a period of time. Accordingly, the pressure may be applied to the user's body by the pressure applying part 32. When the gradually increasing pressure is applied to the user's body by the pressure applying part 32, a blood vessel of the user may be constricted, such that a blood flow rate may decrease or become zero. In some cases, the volume of the pressure applying part 32 may increase before the pressure is applied to the user's body. In these cases, the blood vessel of the user is not constricted, and the blood flow rate does not necessarily decrease or become zero.

Next, the pressure sensor 36 measures a pressure during a period of time, and the controller 38 generates a first pressure signal PSS1 based on a pressure measurement value (S30).

Referring further to FIG. 5, when the gradually increasing pressure is applied to the user's body, the pressure sensor 36 may measure the pressure applied to the user's body. In addition, the controller 38 may generate the first pressure signal PSS1, which is the pressure measurement value during a period of time measured by the pressure sensor 36.

The first pressure signal PSS1 includes information on the pressure applied to the user's body by the pressure applying part 32. For example, when the volume of the pressure applying part 32 gradually increases, the first pressure signal PSS1 may be calculated based on the first pressure signal for a period of time including a first non-measurement section TT1 and a second non-measurement section TT3. In some cases, during the first non-measurement section TT1 the pressure measurement value is constant. In some cases, during the measurement section TT2 the pressure measurement value varies. For example, during the measurement section TT2 the present measurement value gradually increases. In some cases, the measurement section TT2 is a section between a first point in time F1 and a second point in time F2, where a gradient of the first pressure signal PSS1 varies between F1 and F2. The volume of the pressure applying part 32 gradually increases to apply pressure to the user's body. In some cases, before the pressure measurement device 30 comes into contact with the user's body, even though the volume of the pressure applying part 32 increases, the pressure measurement value may not increase. In addition, after the first point in time F1 at which the pressure applying part 32 comes into contact with the user's body, as the volume of the pressure applying part 32 gradually increases, the pressure may be gradually applied to the user's body. Accordingly, the pressure measurement value of the first pressure signal PSS1 measured by the pressure sensor 36 may increase during the measurement section TT2. In addition, after the second point in time F2 at which the measurement section TT2 ends, the pressure applied to the user's body may no longer increase during the second non-measurement section TT3.

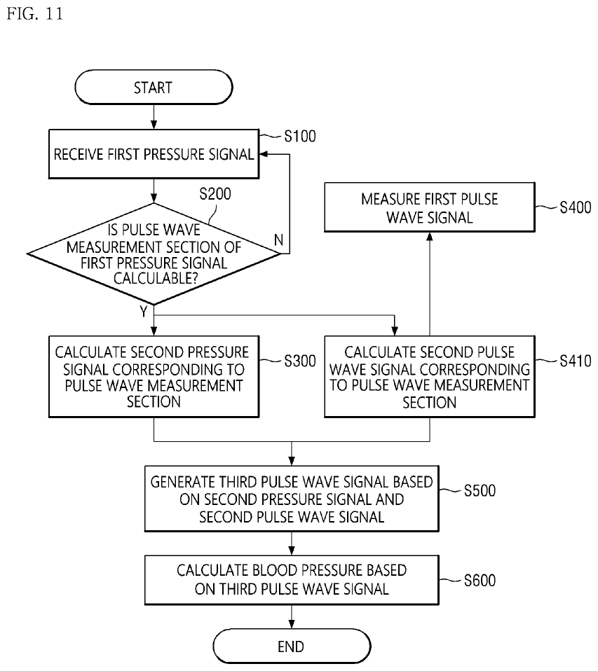

When the pressure measurement value of the first pressure signal PSS1 increases, a blood vessel of the user may be contracted, such that a blood flow rate may decrease or become zero. That is, blood pressure information may be calculated by measuring information on a pulse wave during the measurement section TT2 in which the blood vessel of the user is occluded. A method of calculating blood pressure information will be described later with reference to FIG. 11 and the drawings after FIG. 11.

According to some embodiments, the pressure measurement device 30 transmits the first pressure signal PSS1 to the display device 1 (S40). For example, the controller 38 may transmit the first pressure signal PSS1 to the communication module 35, and the communication module 35 may transmit the first pressure signal PSS1 to the communication unit 160 of the display device 1. Accordingly, the display device 1 may obtain information on the pressure applied to the user's body during a period of time by the pressure measurement device 30. A method in which the display device 1 calculates blood pressure information based on pressure information will be described later with reference to FIG. 11 and the drawings after FIG. 11.

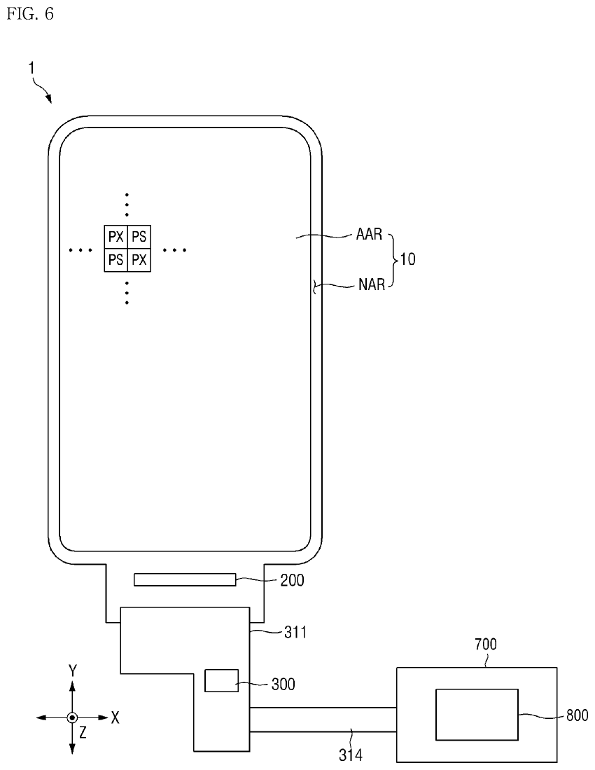

FIG. 6 is a plan view illustrating a display device according to an embodiment.

Referring to FIG. 6, the display device 1 may include a display panel 10, a display driver 200, a circuit board 311, a pulse wave sensing circuit 300, a main circuit board 700, and a main processor 800.

The display panel 10 may include an active area AAR and a non-active area NAR.

The active area AAR includes a display area in which a screen is displayed. The active area AAR may completely overlap the display area. A plurality of pixels PX displaying an image may be disposed in the display area. Each pixel PX may include a light emitting unit.

The active area AAR further includes a light sensing area. The light sensing area is an area sensing light. For example, the light sensing area may be configured to sense an amount of external light of a wavelength, or the like. For example, the light sensing area may sense an incident light. The light sensing area may overlap the display area. In an embodiment, the light sensing area may completely overlap the active area AAR in a plan view. In this case, the light sensing area and the display area may be the same as each other. In another embodiment, the light sensing area may be disposed only in a portion of the active area AAR. For example, the light sensing area may be disposed only in a limited area required for fingerprint recognition. In this case, the light sensing area may overlap a portion of the display area, but may not overlap another portion of the display area.

A plurality of photo-sensors PS responding to light may be disposed in the light sensing area.

The non-active area NAR may be disposed around the active area AAR. The display driver 200 may be disposed in the non-active area NAR. The display driver 200 may drive the plurality of pixels PX and/or the plurality of photo-sensors PS. The display driver 200 may output signals and voltages for driving the display panel 10. The display driver 200 may be formed as an integrated circuit (IC) and be mounted on the display panel 10. Signal lines for transferring signals between the display driver 200 and the active area AAR may be further disposed in the non-active area NAR. In another example, the display driver 200 may be mounted on the circuit board 311.

The circuit board 311 may be attached to one end of the display panel 10 using an anisotropic conductive film (ACF). Lead lines of the circuit board 311 may be electrically connected to pad parts of the display panel 10. The circuit board 311 may be a flexible printed circuit board or a flexible film such as a chip on film.

The pulse wave sensing circuit 300 may be disposed on the circuit board 311. The pulse wave sensing circuit 300 may be formed as an integrated circuit and be attached to an upper surface of the circuit board 311. The pulse wave sensing circuit 300 may be connected to a display layer of the display panel 10. The pulse wave sensing circuit 300 may sense a photocurrent generated by photocharges incident on the plurality of photo-sensors PS of the display panel 10. The pulse wave sensing circuit 300 may identify a pulse wave of a user based on the photocurrent.

The main circuit board 700 may be a printed circuit board or a flexible printed circuit board.

The main circuit board 700 may include the main processor 800.

The main processor 800 may control all functions of the display device 1. For example, the main processor 800 may output digital video data to the display driver 200 through the circuit board 311 so that the display panel 10 displays an image. In addition, the main processor 800 may receive touch data from a touch driving circuit, determine touch coordinates of the user, and then execute an application indicated by an icon displayed on the touch coordinates of the user.

The main processor 800 may calculate a pulse wave signal PPG reflecting a change of blood pressure depending on a heartbeat according to an optical signal input from the pulse wave sensing circuit 300. In addition, the main processor 800 may calculate a pressure measurement value of the user according to a first pressure signal PSS1 input from the pressure measurement device 30. In addition, the main processor 800 may calculate a blood pressure of the user based on the pulse wave signal PPG and a pressure signal.

The main processor 800 may be an application processor formed as an integrated circuit, a central processing unit, or a system chip.

In addition, a mobile communication module capable of transmitting and receiving wireless signals to and from at least one of a base station, an external terminal, and a server over a mobile communication network may be further mounted on the main circuit board 700. The wireless signal may include various types of data according to transmission/reception of a voice signal, a video call signal, or a text/multimedia message.

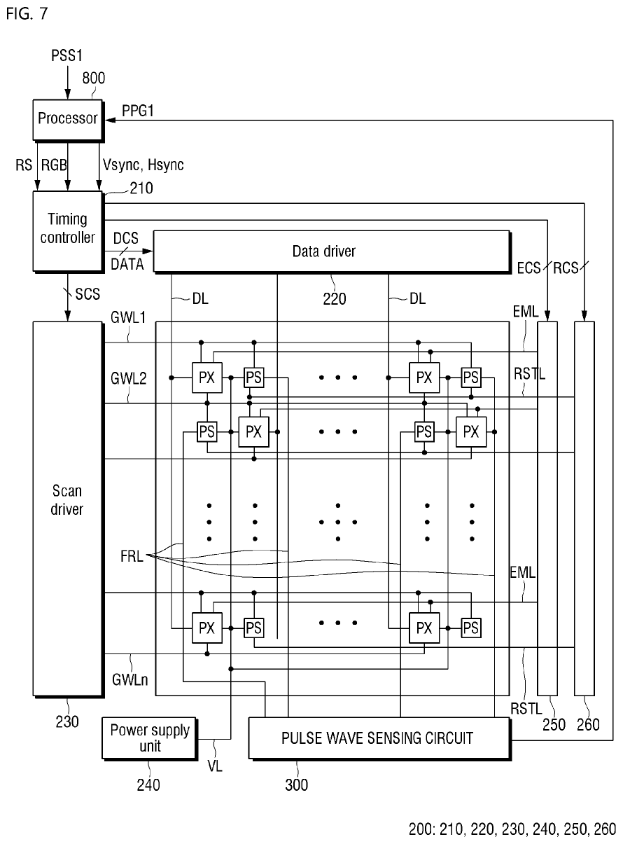

FIG. 7 is a block diagram illustrating the display device according to an embodiment.

The display device 1 according to the present embodiment includes the main processor 800, the display driver 200, and the pulse wave sensing circuit 300.

Referring to FIG. 7, the main processor 800 supplies an image signal RGB supplied from the outside and a plurality of control signals to a timing controller 210. The main processor 800 may further include a graphics processing unit (hereinafter, referred to as a “GPU”) providing graphics for the image signal RGB provided from the outside. The image signal RGB is an image source on which graphics processing has been completed by the GPU, and may be provided to the timing controller 210.

The main processor 800 supplies a sensing timing signal RS to the display driver 200. The sensing timing signal RS is defined as a signal for controlling a sensing timing of the photo-sensor PS. The sensing timing of the photo-sensor PS is controlled according to the sensing timing signal RS. A detailed description of the sensing timing signal RS will be provided later with reference to FIG. 11.

In the display device 1 according to the present embodiment, the plurality of control signals may include a vertical synchronization signal Vsync, a horizontal synchronization signal Hsync, a clock signal, an enable signal, and the like.

The vertical synchronization signal Vsync defines respective frame periods. The vertical synchronization signal Vsync includes a high period and a low period for each cycle, and the cycle of the vertical synchronization signal Vsync corresponds to a frame frequency of each period. In addition, the horizontal synchronization signal Hsync defines horizontal cycles within one frame period. The horizontal synchronization signal Hsync includes a high period and a low period for each cycle, and the cycle of the horizontal synchronization signal Hsync corresponds to each of horizontal cycles. A cycle may refer to the repetitive pattern of a signal. For example, a cycle refers to the complete repetition of a signal, such as the vertical synchronization signal Vsync or the horizontal synchronization signal Hsync. For example, the vertical synchronization signal Vsync alternates between high and low periods for each complete repetition, and this repetition of the vertical synchronization signal corresponds to the frame frequency of each period. Similarly, the horizontal synchronization signal Hsync alternates between high and low periods for each complete repetition, and this repetition of the horizontal synchronization signal corresponds to each individual horizontal repetition within the frame period. In this context, the cycle of a signal is the duration between two repeated patterns of the same signal, in the case of the Vsync and Hsync. For example, each cycle is the duration between two consecutive high-low or low-high pattern of the signal.

The display driver 200 may generate signals and voltages for driving the pixels PX and the photo-sensors PS of the display panel 10. The display driver 200 may be formed as an integrated circuit (IC) and attached to a circuit board in a chip on film (COF) manner, but might not be necessarily limited thereto, and may also be attached onto the non-active area NAR of the display panel 10 in a chip on glass (COG) manner, a chip on plastic (COP) manner, or an ultrasonic bonding method.

The panel driver 200 includes a data driver 220 driving the pixels PX of the display panel 10, a scan driver 230 driving the pixels PX and the photo-sensors PS, and the timing controller 210 controlling driving timings of the data driver 220 and the scan driver 230. In addition, the panel driver may further include a power supply unit 240, an emission driver 250, and a reset driver 260.

The timing controller 210 receives the image signal RGB, the vertical synchronization signal Vsync, and the horizontal synchronization signal Hsync supplied from the outside of the display device 1. The timing controller 210 may output image data DATA and a data control signal DCS to the data driver 220. In addition, the timing controller 210 may generate a scan control signal SCS for controlling an operation timing of the scan driver 230 and an emission control signal ECS for controlling an operation timing of the emission driver 250. For example, the timing controller 210 may generate the scan control signal SCS and the emission control signal ECS, output the scan control signal SCS to the scan driver 230 through a scan control line, and output the emission control signal ECS to the emission driver 250 through an emission control line.

In addition, the timing controller 210 receives the sensing timing signal RS from the main processor 800. The timing controller 210 may generate a reset control signal RCS for controlling an operation timing of the reset driver 260 based on the sensing timing signal RS. For example, the timing controller 210 may generate the reset control signal RCS and output the reset control signal RCS to the reset driver 260 through a reset control line.

The timing controller 210 drives the pixels PX and the photo-sensors PS of the display panel 10. For example, the timing controller 210 outputs the scan control signal SCS, the data control signal DCS, the emission control signal ECS, and the reset control signal RCS.

The data driver 220 may convert the image data DATA into analog data voltages and output the analog data voltages to data lines DL. The data driver 220 may convert the image data DATA into the analog data voltages.

The scan driver 230 may generate scan write signals according to the scan control signal SCS, and sequentially output the scan write signals to scan write lines GWL1 to GWLn. For example, the scan driver 230 receives the scan control signal SCS according to a first frame frequency, and outputs a scan write signal according to the first frame frequency.

The power supply unit 240 may generate a first driving voltage and supply the first driving voltage to a driving voltage line VL, and may generate a second driving voltage and supply the second driving voltage to the driving voltage line VL. The driving voltage line VL may include a first driving voltage line and a second driving voltage line. The first driving voltage may be a high potential voltage for driving light emitting elements and photoelectric conversion elements, and the second driving voltage may be a low potential voltage for driving the light emitting elements and the photoelectric conversion elements. That is, the first driving voltage may have a higher potential than the second driving voltage.

The emission driver 250 may generate emission signals according to the emission control signal ECS and sequentially output the emission signals to emission lines EML. For example, the emission driver 250 receives the emission control signal ECS according to the first frame frequency, and outputs an emission signal according to the first frame frequency. Meanwhile, it has been illustrated in FIG. 7 that the emission driver 250 exists separately from the scan driver 230, but the present disclosure might not be necessarily limited thereto, and the emission driver 250 may also be included in the scan driver 230.

The reset driver 260 may generate reset signals according to the reset control signal RCS and sequentially output the reset signals to reset lines RSTL. For example, the reset driver 260 receives the reset control signal RCS according to the first frame frequency, and outputs a reset signal according to the first frame frequency.

The pulse wave sensing circuit 300 may be connected to the respective photo-sensors PS through light sensing lines FRL, and may receive light sensing signals of the respective photo-sensors PS (e.g., currents flowing to the photo-sensors PS) and calculate a pulse wave signal. The pulse wave sensing circuit 300 may be formed as an integrated circuit (IC) and attached to a display circuit board in a chip on film (COF) manner, but might not be necessarily limited thereto, and may also be attached onto the non-active area NAR of the display panel 10 in a chip on glass (COG) manner, a chip on plastic (COP) manner, or an ultrasonic bonding method.

The pulse wave sensing circuit 300 may generate a first pulse wave signal PPG1 according to magnitudes of the currents sensed by the respective photo-sensors PS and transmit the first pulse wave signal PPG1 to the main processor 800, and the main processor 800 may calculate blood pressure information of the user by analyzing the first pulse wave signal PPG1 and a first pressure signal measured by the pressure measurement device 30. In summary, the main processor 800 applies the sensing timing signal RS to the display driver 200, and the display driver 200 controls the sensing timing of the photo-sensor PS based on the sensing timing signal RS. Accordingly, the pulse wave sensing circuit 300 may output the first pulse wave signal PPG1 sensed by the photo-sensor PS to the main processor 800.

The display panel 10 further includes a plurality of pixels PX, a plurality of photo-sensors PS, a plurality of scan write lines GWL1 to GWLn connected to the plurality of pixels PX and the plurality of photo-sensors PS, a plurality of data lines DL and a plurality of emission lines EML connected to the plurality of pixels PX, and a plurality of light sensing lines FRL and a plurality of reset lines RSTL connected to the plurality of photo-sensors PS.

Each of the plurality of pixels PX may include a light emitting element and a plurality of transistors controlling an amount of light emitted from the light emitting element. Each of the plurality of pixels PX may be connected to at least one of the scan write lines GWL1 to GWLn, any one of the data lines DL, at least one of the emission lines EML, and the driving voltage line VL.

Each of the plurality of photo-sensors PS may include a photoelectric conversion element and a plurality of transistors controlling an amount of light received by the photoelectric conversion element. Each of the plurality of photo-sensors PS may be connected to any one of the scan write lines GWL1 to GWLn, any one of the reset lines RSTL, at least one of the light sensing lines FRL, and the driving voltage line VL.

The plurality of scan write lines GWL1 to GWLn may connect the scan driver 230 to the plurality of pixels PX and the plurality of photo-sensors PS, respectively. The plurality of scan write lines GWL1 to GWLn may provide the scan write signals output from the scan driver 230 to the plurality of pixels PX and the plurality of photo-sensors PS, respectively.

The plurality of data lines DL may connect the data driver 220 to the plurality of pixels PX, respectively. The plurality of data lines DL may provide the image data output from the data driver 220 to the plurality of pixels PX, respectively.

The plurality of emission lines EML may connect the emission driver 250 to the plurality of pixels PX, respectively. The plurality of emission lines EML may provide the emission signals output from the emission driver 250 to the plurality of pixels PX, respectively.

The plurality of reset lines RSTL may connect the reset driver 260 to the plurality of photo-sensors PS, respectively. The plurality of reset lines RSTL may provide the reset signals output from the reset driver 260 to the plurality of photo-sensors PS, respectively. The plurality of light sensing lines FRL may connect the plurality of photo-sensors PS to the pulse wave sensing circuit 300, respectively. The plurality of light sensing lines FRL may provide the photocurrent output from each of the plurality of photo-sensors PS to the pulse wave sensing circuit 300. Accordingly, the pulse wave sensing circuit 300 may generate a pulse wave signal of the user or sense a fingerprint of the user.

A plurality of driving voltage lines VL may connect the power supply unit 240 to the plurality of pixels PX and the plurality of photo-sensors PS, respectively. The plurality of driving voltage lines VL may provide the first driving voltage or the second driving voltage from the power supply unit 240 to the plurality of pixels PX and the plurality of photo-sensors PS, respectively.

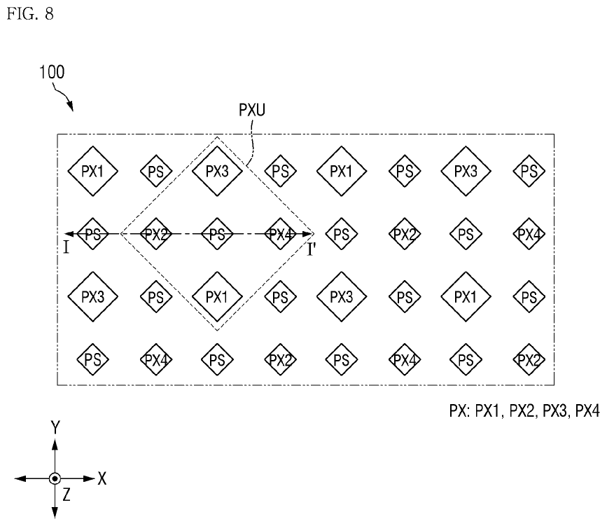

FIG. 8 is a plan layout view of pixels and photo-sensors of a display panel according to an embodiment.

Referring to FIG. 8, a plurality of pixels PX and a plurality of photo-sensors PS may be repeatedly disposed in the display panel 10.

The plurality of pixels PX: PX1, PX2, PX3, and PX4 may include first pixels PX1, second pixels PX2, third pixels PX3, and fourth pixels PX4. For example, the first pixel PX1 may emit light of a red wavelength, the second pixel PX2 and the fourth pixel PX4 may emit light of a green wavelength, and the third pixel PX3 may emit light of a blue wavelength. The plurality of pixels PX may include a plurality of emission areas emitting light, respectively. The plurality of photo-sensors PS may include a plurality of light sensing areas sensing light incident thereon.

The first pixels PX1, the second pixels PX2, the third pixels PX3, and the fourth pixels PX4 and the plurality of photo-sensors PS may be alternately arranged in the first direction X and the second direction Y. In an embodiment, the first pixels PX1 and the third pixels PX3 may be alternately arranged while forming a first row along the first direction X, and the second pixels PX2 and the fourth pixels PX4 may be repeatedly arranged along the first direction X in a second row adjacent to the first row. Pixels PX belonging to the first row may be staggered with pixels PX belonging to the second row in the first direction X. Arrangements of the first row and the second row may be repeated up to an n-th row.

The photo-sensors PS may be disposed between the first pixels PX1 and the third pixels PX3 forming the first row and be disposed to be spaced apart from each other. The first pixels PX1, the photo-sensors PS, and the third pixels PX3 may be alternately arranged along the first direction X. The photo-sensors PS may be disposed between the second pixels PX2 and the fourth pixels PX4 forming the second row and be disposed to be spaced apart from each other. The second pixels PX2, the photo-sensors PS, and the fourth pixels PX4 may be alternately arranged along the first direction X. The number of photo-sensors PS in the first row may be the same as the number of photo-sensors PS in the second row. Arrangements of the first row and the second row may be repeated up to an n-th row.

In another example, the photo-sensors PS may be disposed between the second pixels PX2 and the fourth pixels PX4 forming the second row, and may not be disposed between the first pixels PX1 and the third pixels PX3 forming the first row. That is, the photo-sensors PS may not be disposed in the first row.

Sizes of emission areas of the respective pixels PX may be different from each other. Sizes of emission areas of the second pixel PX2 and the fourth pixel PX4 may be smaller than those of emission areas of the first pixel PX1 or the third pixel PX3. It has been illustrated in FIG. 8 that the respective pixels PX have a rhombic shape, but the present disclosure might not be necessarily limited thereto, and the respective pixels PX have may have a rectangular shape, an octagonal shape, a circular shape, or other polygonal shapes.

One pixel unit PXU may include one first pixel PX1, one second pixel PX2, one third pixel PX3, and one fourth pixel PX4. The pixel unit PXU refers to a group of color pixels capable of expressing a gradation.

FIG. 9 is a circuit diagram illustrating a pixel and a photo-sensor according to an embodiment in detail.

Referring to FIG. 9, a first pixel PX1 may be connected to a scan start line GIL, a scan control line GCL, a first scan write line GWL1, a second scan write line GWL2, an emission line EML, and a data line DL. In addition, each of the pixels PX may be connected to a first driving voltage line VDDL to which the first driving voltage is applied, a second driving voltage line VSSL to which the second driving voltage is applied, a first initialization voltage line to which a first initialization voltage Vint1 is applied, and a second initialization voltage line to which a second initialization voltage Vint2 is applied.

A first photo-sensor PS1 may be connected to a first scan write line GWL1, a reset line RSTL, and a light sensing line FRL. In addition, each of the photo-sensors PS may be connected to the second driving voltage line VSSL to which the second driving voltage is applied, a reset voltage line to which a reset voltage Vrst is applied, and a second initialization voltage line to which a second initialization voltage Vint2 is applied.

The first pixel PX1 may include a plurality of transistors, a light emitting element EL, and at least one capacitor Cst. The plurality of transistors may include first to seventh transistors T1, T2, T3, T4, T5, T6, and T7. Among them, the first transistor T1 may be a driving transistor, and the second to seventh transistors T2, T3, T4, T5, T6, and T7 may be transistors serving as switch elements turned on or off according to scan write signals applied to their gate electrodes.

The first transistor T1 may include a gate electrode, a first electrode, and a second electrode. The first transistor T1 may control a source-drain current lsd (hereinafter, referred to as a “driving current lsd”) according to a data voltage applied to the gate electrode. The driving current lsd flowing through a channel of the first transistor T1 is proportional to the square of a difference between a voltage between a source electrode and the gate electrode of the first transistor T1 and an absolute value of a threshold voltage (Vth) of the first transistor T1 as represented in Equation (1).

lsd=k′×(Vsg−|Vth|)² Equation (1)

In Equation 1, k′ refers to a proportional coefficient determined by a structure and physical properties of the first transistor T1, Vsg refers to a source-gate voltage of the first transistor T1, and Vth refers to the threshold voltage of the first transistor T1.

The gate electrode of the first transistor T1 may be connected to the first electrode of the third transistor T3 and one electrode of the capacitor Cst, the first electrode of the first transistor T1 may be connected to a second electrode of the second transistor T2 and a second electrode of the fifth transistor T5, and the second electrode of the first transistor T1 may be connected to a second electrode of the third transistor T3 and a first electrode of the sixth transistor T6.

The light emitting element EL emits light according to the driving current lsd. An amount of light emitted from the light emitting element EL may be proportional to the driving current lsd.

The light emitting element EL may be an organic light emitting diode including an anode electrode, a cathode electrode, and an organic light emitting layer disposed between the anode electrode and the cathode electrode. Alternatively, the light emitting element EL may be an inorganic light emitting diode including an anode electrode, a cathode electrode, and an inorganic light emitting layer disposed between the anode electrode and the cathode electrode or may be a quantum dot light emitting element EL including an anode electrode, a cathode electrode, and a quantum dot light emitting layer disposed between the anode electrode and the cathode electrode. In addition, the light emitting element EL may be a micro light emitting diode.

The anode electrode of the light emitting element EL may be connected to a second electrode of the sixth transistor T6 and a second electrode of the seventh transistor T7, and the cathode electrode of the light emitting element EL may be connected to the second driving voltage line VSSL.

The second transistor T2 may be turned on by a scan write signal of the first scan write line GWL1 to connect the first electrode of the first transistor T1 and the data line DL to each other. A gate electrode of the second transistor T2 may be connected to the first scan write line GWL1, a first electrode of the second transistor T2 may be connected to the data line DL, and the second electrode of the second transistor T2 may be connected to the first electrode of the first transistor T1.

The third transistor T3 may be turned on by a scan write signal of the scan control line GCL to connect the gate electrode and the second electrode of the first transistor T1 to each other. That is, when the third transistor T3 is turned on, the gate electrode and the second electrode of the first transistor T1 are connected to each other, and thus, the first transistor T1 may be driven as a diode. A gate electrode of the third transistor T3 may be connected to the scan control line GCL, the first electrode of the third transistor T3 may be connected to the second electrode of the first transistor T1, and the second electrode of the third transistor T3 may be connected to the gate electrode of the first transistor T1.

The fourth transistor T4 may be turned on by a scan write signal of the scan start line GIL to connect the gate electrode of the first transistor T1 and the second initialization voltage line to each other. In this case, the gate electrode of the first transistor T1 may be discharged to the second initialization voltage Vint2 of the second initialization voltage line. A gate electrode of the fourth transistor T4 may be connected to the scan start line GIL, a first electrode of the fourth transistor T4 may be connected to the second initialization voltage line, and a second electrode of the fourth transistor T4 may be connected to the gate electrode of the first transistor T1.

The fifth transistor T5 may be turned on by an emission signal of the emission line EML to connect the first electrode of the first transistor T1 and the first driving voltage line VDDL to each other. A gate electrode of the fifth transistor T5 may be connected to the emission line EML, a first electrode of the fifth transistor T5 may be connected to the first driving voltage line VDDL, and the second electrode of the fifth transistor T5 may be connected to the first electrode of the first transistor T1.

The sixth transistor T6 may be turned on by the emission signal of the emission line EML to connect the second electrode of the first transistor T1 and the anode electrode of the light emitting element EL to each other. A gate electrode of the sixth transistor T6 may be connected to the emission line EML, the first electrode of the sixth transistor T6 may be connected to the second electrode of the first transistor T1, and the second electrode of the sixth transistor T6 may be connected to the anode electrode of the light emitting element EL.

When both the fifth transistor T5 and the sixth transistor T6 are turned on, the driving current lsd may be supplied to the light emitting element EL.

The seventh transistor T7 may be turned on by a scan write signal of the second scan write line GWL2 to connect the first initialization voltage line and the anode electrode of the light emitting element EL to each other. In this case, the anode electrode of the light emitting element EL may be discharged to the first initialization voltage Vint1. A gate electrode of the seventh transistor T7 may be connected to the second scan write line GWL2, a first electrode of the seventh transistor T7 may be connected to the first initialization voltage line, and the second electrode of the seventh transistor T7 may be connected to the anode electrode of the light emitting element EL.

The capacitor Cst may be formed between the gate electrode of the first transistor T1 and the first driving voltage line VDDL. One electrode of the capacitor Cst may be connected to the gate electrode of the first transistor T1, and the other electrode of the capacitor Cst may be connected to the first driving voltage line VDDL. Therefore, the capacitor Cst may maintain a potential difference between the gate electrode of the first transistor T1 and the first driving voltage line VDDL.

Each of the photo-sensors PS includes a first photo-sensor PS1 connected to the first scan write line GWL1. The first photo-sensor PS1 may be repeatedly arranged along a plurality of rows in the display panel 10.

The first photo-sensor PS1 may include a plurality of sensing transistors and a photoelectric conversion element PD. The plurality of sensing transistors may include first to third sensing transistors LT1, LT2, and LT3. The first photo-sensor PS1 may include a first node N1 between the first sensing transistor LT1, the third sensing transistor LT3, and the photoelectric conversion element PD and a second node N2 between the second driving voltage line VSSL and the photoelectric conversion element PD. The first sensing transistor LT1 may be a driving transistor, and the second and third sensing transistors LT2 and LT3 may be transistors serving as switch elements turned on or off according to a reset signal and a scan write signal applied to their gate electrodes.

When a plurality of light emitting elements EL and a plurality of photoelectric conversion elements PD are disposed in one display panel 10, voltage lines or signal lines for driving the light emitting elements EL may be used in common in driving the photoelectric conversion elements PD. That is, it is minimized that voltage lines or signal lines for driving the plurality of photoelectric conversion elements PD are additionally disposed in the display panel 10, such that resolution of the display panel 10 may be secured and a bezel area of the display panel 10 may be minimized. In an example embodiment of the present inventive concept, a signal line connected to the gate electrode of the second transistor T2 of the pixel PX may be used in common with a signal line connected to a gate electrode of the second sensing transistor LT2 of the photo-sensor PS. That is, the gate electrode of the second transistor T2 and the gate electrode of the second sensing transistor LT2 may be connected to the first scan write line GWL1. In another example, the second driving voltage line VSSL may be a common voltage line connected to the cathode electrode of the light emitting element EL and a cathode electrode of the photoelectric conversion element PD. As still another example, the second initialization voltage line applying the second initialization voltage Vint2 may be a common voltage line connected to a second electrode of the first sensing transistor LT1 of the photo-sensor PS and the second electrode of the fourth transistor T4.

Each of the photoelectric conversion elements PD may a light receiving diode including an anode electrode, a cathode electrode, and a photoelectric conversion layer disposed between the anode electrode and the cathode electrode. Each of the photoelectric conversion elements PD may convert light incident from the outside into an electrical signal. The photoelectric conversion element PD may be a light receiving diode or a phototransistor made of a pn-type or pin-type inorganic material. Alternatively, the photoelectric conversion element PD may be an organic light receiving diode including an electron donating material generating donor ions and an electron accepting material generating acceptor ions.

The anode electrode of the photoelectric conversion element PD may be connected to the first node N1, and the cathode electrode of the photoelectric conversion element PD may be connected to the second node N2.

The photoelectric conversion element PD may generate photocharges when it is exposed to external light, and the generated photocharges may be accumulated in the anode electrode of the photoelectric conversion element PD. In this case, a voltage of the first node N1 electrically connected to the anode electrode may increase. When the photoelectric conversion element PD and the light sensing line FRL are connected to each other according to the turn-on of the first and second sensing transistors LT1 and LT2, a current may flow to the light sensing line FRL in proportion to the voltage of the first node N1 in which the charges are accumulated.

The first sensing transistor LT1 may be turned on by the voltage of the first node N1 applied to a gate electrode thereof to connect the second initialization voltage line and a first electrode of the second sensing transistor LT2 to each other. In this case, the second electrode of the second sensing transistor LT2 may be discharged to the second initialization voltage Vint2. The gate electrode of the first sensing transistor LT1 may be connected to the first node N1, a first electrode of the first sensing transistor LT1 may be connected to the second initialization voltage line, and a second electrode of the first sensing transistor LT1 may be connected to the first electrode of the second sensing transistor LT2. The first sensing transistor LT1 may be a source follower amplifier generating a source-drain current in proportion to a quantity of charges of the first node N1 input to the gate electrode. Meanwhile, the first electrode of the first sensing transistor LT1 may also be connected to the first driving voltage line VDDL or the first initialization voltage line.

The second sensing transistor LT2 may be turned on by a scan write signal of the first scan write line GWL1 to connect the second electrode of the first sensing transistor LT1 and the light sensing line FRL to each other. The light sensing line FRL may transfer a light sensing signal to the pulse wave sensing circuit 300 (see FIG. 7). The gate electrode of the second sensing transistor LT2 may be connected to the first scan write line GWL1, the first electrode of the second sensing transistor LT2 may be connected to the second electrode of the first sensing transistor LT1, and a second electrode of the second sensing transistor LT2 may be connected to the light sensing line FRL.

The third sensing transistor LT3 may be turned on by a reset signal of the reset line RSTL to reset the voltage of the first node N1 to the reset voltage Vrst. A gate electrode of the third sensing transistor LT3 may be connected to the reset line RSTL, a first electrode of the third sensing transistor LT3 may be connected to the reset voltage line, and a second electrode of the third sensing transistor LT3 may be connected to the first node N1. When the reset driver outputting the reset signal of the reset line RSTL is omitted, the third sensing transistor LT3 may be turned on by the scan write signal.

When the first electrode of each of the first to seventh transistors T1, T2, T3, T4, T5, T6, and T7 and the first to third sensing transistors LT1, LT2, and LT3 is a source electrode, the second electrode of each of the first to seventh transistors T1, T2, T3, T4, T5, T6, and T7 and the first to third sensing transistors LT1, LT2, and LT3 may be a drain electrode. Alternatively, when the first electrode of each of the first to seventh transistors T1, T2, T3, T4, T5, T6, and T7 and the first to third sensing transistors LT1, LT2, and LT3 is a drain electrode, the second electrode of each of the first to seventh transistors T1, T2, T3, T4, T5, T6, and T7 and the first to third sensing transistors LT1, LT2, and LT3 may be a source electrode.

An active layer of each of the first to seventh transistors T1, T2, T3, T4, T5, T6, and T7 and the first to third sensing transistors LT1, LT2, and LT3 may be made of any one of polysilicon, amorphous silicon, and an oxide semiconductor.

In an example embodiment of the present inventive concept, the first and second transistors T1 and T2, the fifth to seventh transistors T5, T6, and T7, and the first and second sensing transistors LT1 and LT2 may be P-type transistors. In this case, an active layer of each of the first and second transistors T1 and T2, the fifth to seventh transistors T5, T6, and T7, and the first and second sensing transistors LT1 and LT2 may be made of polysilicon. In addition, each of the third transistor T3, the fourth transistor T4, and the third sensing transistor LT3 may be an N-type transistor of which an active layer is made of an oxide semiconductor.

However, embodiments are not limited thereto, and each of the first to seventh transistors T1, T2, T3, T4, T5, T6, and T7, and the first to third sensing transistors LT1, LT2, and LT3 may be a P-type transistor. In another example, the first to third sensing transistors LT1, LT2, and LT3 may be formed as P-type transistors.

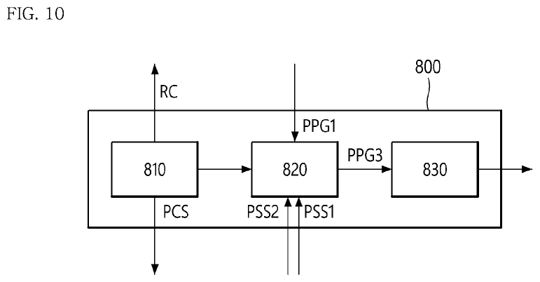

FIG. 10 is a block diagram illustrating a main processor according to an embodiment.

The main processor 800 includes a control signal generator 810, a pulse wave calculator 820, and a blood pressure calculator 830.

The control signal generator 810 may generate the pressure control signal PCS for controlling the pressure measurement device 30 to apply a pressure. The control signal generator 810 may output the pressure control signal PCS to the pressure measurement device 30. Specifically, the control signal generator 810 may apply the pressure control signal PCS to the communication unit 160, and the communication unit 160 may output the pressure control signal PCS to the communication module 35 of the pressure measurement device 30. Accordingly, the controller 38 of the pressure measurement device 30 may receive the pressure control signal PCS from the communication module 35. The controller 38 may gradually apply the pressure to the user's body based on the pressure control signal PCS.

The control signal generator 810 may generate the sensing timing signal RC for controlling a timing at which the photo-sensor PS senses light. The control signal generator 810 may apply the sensing timing signal RC to the photo-sensor PS. Specifically, the control signal generator 810 applies the sensing timing signal RC to the timing controller 210, and the timing controller 210 applies the reset control signal RCS to the reset driver 260 based on the sensing timing signal RC. Accordingly, when the pressure measurement device 30 applies the pressure, the photo-sensor PS may sense light.

In some cases, during a section corresponding to the measurement section, the main processor 800 extracts the first pressure signal to calculate a second pressure signal and extracts the first pulse wave signal to calculate a second pulse wave signal. For example, the pulse wave calculator 820 may be configured to extract the first pressure signal and the first pulse wave signal. The pulse wave calculator 820 may receive the first pressure signal PSS1 having a pressure measurement value during a period of time from the pressure measurement device 30. Accordingly, the pulse wave calculator 820 may calculate the measurement section TT2 in the first pressure signal PSS1. For example, the pulse wave calculator 820 may determine any one of a plurality of measurement sections of the first pressure signal PSS1 as the measurement section TT2. In this case, the pulse wave calculator 820 may determine the measurement section TT2 based on information on the measurement section TT2 of the first pressure signal PSS1 stored in advance in a memory. The pulse wave calculator 820 may calculate a second pressure signal PSS2 corresponding to the measurement section TT2 in the first pressure signal PSS1. In addition, the pulse wave calculator 820 may receive the first pulse wave signal PPG1 from the pulse wave sensing circuit 300.

The pulse wave calculator 820 may receive the first pulse wave signal PPG1 having a pulse wave measurement value during a period of time from the pulse wave sensing circuit 300. The pulse wave calculator 820 may calculate a second pulse wave signal PPG2 corresponding to the measurement section TT2 in the first pulse wave signal PPG1. In addition, the pulse wave calculator 820 may generate a third pulse wave signal PPG3 having a magnitude of a pulse wave signal according to a pressure based on the second pulse wave signal PPG2 and the second pressure signal PSS2. The pulse wave calculator 820 may output the third pulse wave signal PPG3 to the blood pressure calculator 830.

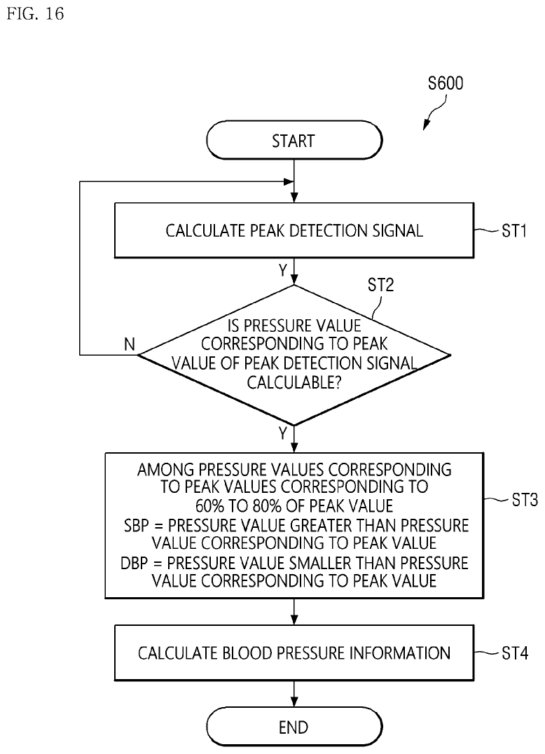

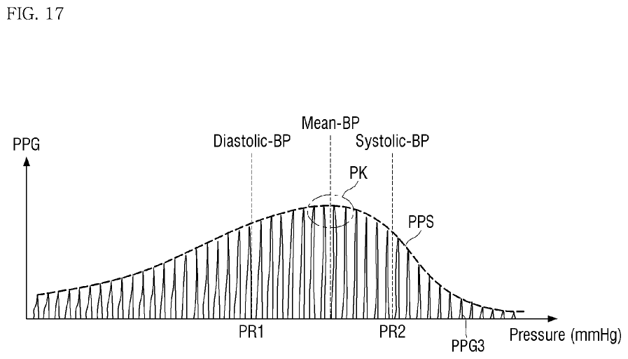

The blood pressure calculator 830 receives the third pulse wave signal PPG3 from the pulse wave calculator 820. The blood pressure calculator 830 may generate a peak detection signal PPS (see FIG. 17) based on data on a cycle and an amplitude of the third pulse wave signal PPG3. The blood pressure calculator 830 may calculate the blood pressure based on a peak value of the generated peak detection signal PPS. This will be described later with reference to FIG. 16.

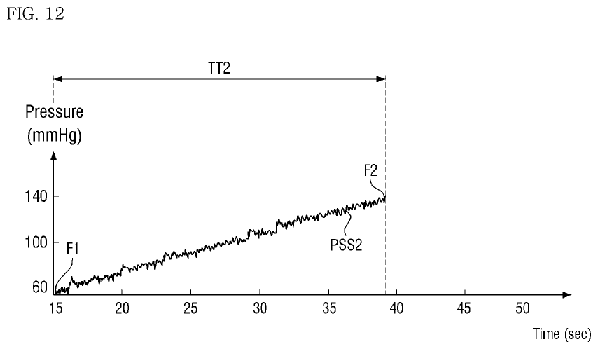

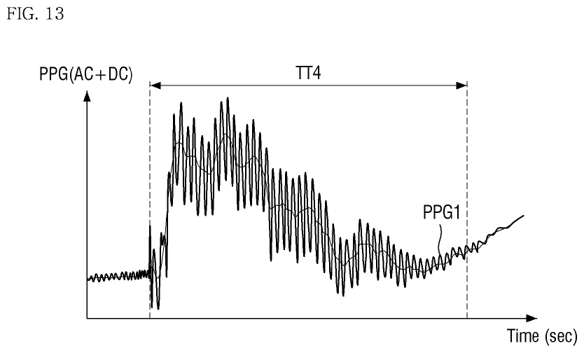

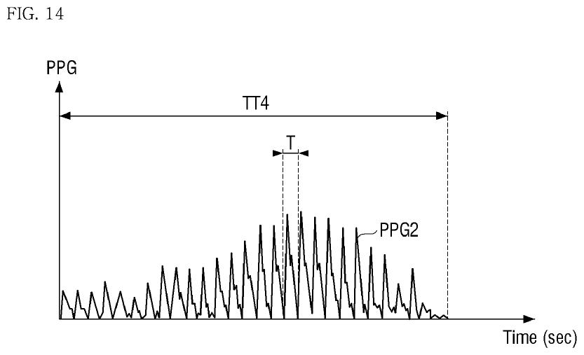

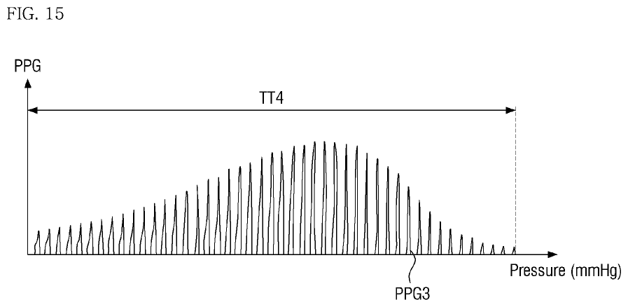

FIG. 11 is a flowchart illustrating a blood pressure calculation method of the blood pressure measurement system according to an embodiment. FIG. 12 is a graph illustrating a second pressure signal during a period of time. FIGS. 13 and 14 are graphs illustrating pulse wave signals during a period of time. FIG. 15 is a graph illustrating a pulse wave signal according to a pressure. Hereinafter, a blood pressure calculation method of the blood pressure measurement system will be described with reference to FIGS. 11 to 15.

Referring to FIG. 11, first, the main processor 800 receives the first pressure signal PSS1 (S100). For example, the communication module 35 of the pressure measurement device 30 may transmit the first pressure signal PSS1 to the communication unit 160 of the display device 1. Accordingly, the main processor 800 may receive the first pressure signal PSS1 from the communication unit 160. The main processor 800 may receive information on the pressure gradually applied to the user's body during a period of time by the pressure measurement device 30.

Next, the main processor 800 determines whether or not the measurement section TT2 of the first pressure signal PSS1 may be calculated (S200).

As described above, the first pressure signal PSS1 according to an embodiment of FIG. 5 includes information on the pressure applied to the user's body by the pressure applying part 32. For example, the first pressure signal PSS1 may be calculated based on the first pressure signal for a period of time including the first non-measurement section TT1 and the second non-measurement section TT3. During the first non-measurement section TT1 the pressure measurement value is constant. During the measurement section TT2 the pressure measurement value varies, for example, gradually increases. Here, the measurement section TT2 is defined as a section between the first point in time F1 and the second point in time F2. In these examples, a gradient of the pressure signal varies between F1 and F2.

In the measurement section TT2, by applying a gradually increasing pressure to the blood vessel of the user, the gradually increasing pressure is applied to the blood vessel. In this case, a volume of the artery of the user may change depending on systolic and diastolic blood pressures of the heart of the user, and a blood pressure may be calculated based on a pressure applied to the artery of the user by the pressure measurement device 30. Meanwhile, the pressure measurement value does not change during a period of time at a pressure equal to or less than a pressure at the first point in time F1 and equal to or more than a pressure at the second point in time F2. This means that there is no change in the pressure applied to the user's body depending on an external pressure.

Accordingly, the main processor 800 may calculate the measurement section TT2 in the first pressure signal PS Sl.

Next, the main processor 800 calculates the second pressure signal PSS2 corresponding to the measurement section TT2 in the first pressure signal PSS1 (S300).

Referring further to FIG. 12, when the main processor 800 calculates the measurement section TT2 in the first pressure signal PSS1, the main processor 800 calculates a signal corresponding to the measurement section TT2 in the first pressure signal PSS. For example, the main processor 800 may calculate the section between the first point in time F1 and the second point in time F2 as the second pulse wave signal. Alternatively, the main processor 800 may calculate a section in which a magnitude of the first pressure signal PSS1 varies during a period of time as the second pulse wave signal.

That is, the second pressure signal PSS2 may be a section in which a pressure that gradually increases during a period of time is applied to the artery of the user.

Meanwhile, the pulse wave sensing circuit measures the first pulse wave signal (S400).

Referring further to FIGS. 13 and 14, in order to generate the first pulse wave signal PPG1, pulse wave information during a period of time is also required together with the pressure data. During systole of the heart, blood ejected from the left ventricle of the heart moves to peripheral tissues, such that a blood volume in the arterial side increases. In addition, during the systole of the heart, red blood cells carry more oxyhemoglobin to the peripheral tissues. During diastole of the heart, there is partial suction of blood from the peripheral tissues toward the heart. In this case, when a peripheral blood vessel is irradiated with light emitted from a display pixel, the irradiated light may be absorbed by the peripheral tissue. Absorbance is dependent on a hematocrit and a blood volume. The absorbance may have a maximum value during the systole of the heart and a minimum value during the diastole of the heart. Since the absorbance is in inverse proportion to an amount of light incident on the photo-sensor PS, absorbance at a corresponding point in time may be estimated through light reception data of the amount of light incident on the photo-sensor PS, and accordingly, as illustrated in FIG. 13, the first pulse wave signal PPG1 value during a period of time may be generated.

The pulse wave information during a period of time reflects the maximum value of the absorbance during the systole of the heart, and reflects the minimum value of the absorbance during the diastole of the heart. In addition, the pulse wave fluctuates according to a heartbeat cycle T. Accordingly, the pulse wave information may reflect a change in blood pressure depending on a heartbeat. Accordingly, the pulse wave sensing circuit 300 may measure the first pulse wave signal PPG1 value according to a pressure applying time. However, the pulse wave signal PPG may include both an alternating current (AC) component and a direct current (DC) component. The main processor 800 may remove the DC component from the pulse wave signal to generate a pulse wave signal plotted according to a magnitude of a time. Accordingly, the main processor 800 may receive the first pulse wave signal PPG1 from the pulse wave sensing circuit 300.

In addition, the main processor 800 calculates the second pulse wave signal corresponding to the measurement section TT2 in the first pulse wave signal (S410).

For example, the first pulse wave signal PPG1 according to an embodiment of FIG. 13 includes data on absorbance of the blood vessel of the user sequentially according to the first non-measurement section TT1, the measurement section TT2, and the second non-measurement section TT3 of the first pressure signal PSS1. In order for the display device 1 to calculate the blood pressure based on the first pressure signal PSS1 and the first pulse wave signal, pulse wave information for a section in which a pressure is directly applied to the blood vessel of the user is required. Accordingly, the pressure is directly applied to the blood vessel of the user during the measurement section TT2 of the first pressure signal PSS1, and thus, the main processor 800 may calculate a pulse wave section TT4 corresponding to the measurement section TT2 in the first pulse wave signal to calculate the second pulse wave signal. That is, the pulse wave section TT4 of the first pulse wave signal may be substantially the same as the measurement section TT2.

Subsequently, the main processor generates the third pulse wave signal based on the second pressure signal and the second pulse wave signal (S500).

Referring further to FIG. 15, the main processor 800 may generate the third pulse wave signal PPG3 based on the second pressure signal PSS2 and the second pulse wave signal PPG2 received from the pulse wave sensing circuit 300. As described above, the second pressure signal PSS2 is a signal having a pressure measurement value according to the measurement section, and the second pulse wave signal PPG2 is a signal of light reception data according to a pulse wave section corresponding to the measurement section. Accordingly, the main processor 800 may generate the third pulse wave signal PPG3 having a magnitude of a pulse wave signal according to a pressure based on the second pressure signal PSS2 and the second pulse wave signal PPG2.

According to some embodiments, the main processor 800 calculates a peak value of the peak detection signal, a pressure value corresponding to the peak value of the peak detection signal, a diastolic blood pressure, a systolic blood pressure, and a mean blood pressure, wherein the diastolic blood pressure is lower than the pressure value corresponding to the peak value and the systolic blood pressure is higher than the pressure value corresponding to the peak value. In some cases, the main processor 800 calculates the blood pressure based on the third pulse wave signal (S600). The main processor 800 may generate a peak detection signal PPS (see FIG. 17) based on peak values of the third pulse wave signal PPG3. In addition, the main processor 800 may calculate the blood pressure information of the user based on the peak value of the peak detection signal PPS (see FIG. 17). This will be described later with reference to FIG. 16.

In a case of the blood pressure measurement method of the blood pressure measurement system according to the present embodiment, the pressure measurement device may measure the pressure applied to the artery of the user, and the display device 1 may measure the pulse wave signal. The blood pressure measurement system according to the present embodiment may more accurately measure the pressure by applying the pressure to the user's body and measuring the pressure applied to the user's body.

In addition, the display device 1 may calculate the measurement section TT2 in the first pressure signal PSS1 to calculate the second pulse wave signal PPG2 and the second pressure signal PSS2. Accordingly, the display device 1 may calculate the blood pressure of the user based on accurate pressure information and pulse wave information to accurately calculate the blood pressure.

FIG. 16 is a flowchart illustrating a method of calculating a blood pressure according to an embodiment. FIG. 17 is a graph illustrating a waveform of a peak detection signal. A method of calculating a blood pressure based on the third pulse wave signal PPG3 will be described with reference to FIGS. 16 and 17.

Referring to FIGS. 16 and 17, first, the main processor 800 generates a peak detection signal PPS (ST1).

According to some embodiments, a cycle of the third pulse wave signal includes a plurality of waveforms, a first waveform and a second waveform of the plurality of waveforms having different amplitudes. The main processor calculates the reflected pulse wave ratio RI using formula RI=Rp/Sp, wherein Sp is a pulse wave contraction value defined by a peak value of the first waveform, and Rp is a reflected pulse wave value defined by a peak value of the second waveform. In some cases, the main processor 800 may calculate the amplitude at each of cycles T of the third pulse wave signal PPG3. In addition, the main processor 800 may generate the peak detection signal PPS having a magnitude of the third pulse wave signal PPG3 based on an amplitude of each of the cycles T of the third pulse wave signal PPG3. For example, the peak detection signal PPS is defined as a signal corresponding to an amplitude of each of the cycles of the third pulse wave signal PPG3. That is, the peak detection signal PPS may be defined as a signal corresponding to a peak value of each of the cycles of the third pulse wave signal PPG3. For example, the third pulse wave signal PPG3 may have one or more amplitudes. The main processor 800 may calculate the peak detection signal PPS including points corresponding to amplitudes of the respective cycles T of the third pulse wave signal PPG3. That is, the generated peak detection signal PPS may be a signal having an amplitude according to a pressure.

Next, the main processor 800 determines whether or not a pressure value corresponding to a peak value PK of the peak detection signal PPS may be calculated (ST2). When a peak of the peak detection signal PPS exists, the main processor 800 may calculate the pressure value corresponding to the peak value PK of the peak detection signal PPS.

Next, the main processor 800 calculates a systolic blood pressure SBP, a diastolic blood pressure DBP, and the like, based on the peak value PK of the peak detection signal PPS (ST3), and calculates blood pressure information (ST4).

The main processor 800 may calculate the diastolic blood pressure DBP lower than the pressure value, the systolic blood pressure SBP higher than the pressure value, and a mean blood pressure according to the pressure value. According to some embodiments, the diastolic blood pressure is smaller than a pressure value corresponding to 60% to 80% of the peak value and the systolic blood pressure is greater than the pressure value corresponding to 60% to 80% of the peak value. For example, the main processor 800 may calculate pressure values corresponding to values corresponding to 60% to 80% of the peak value PK. The main processor 800 may calculate a pressure value smaller than a pressure value corresponding to the peak value PK among the pressure values as a first pressure value PR1. In addition, the main processor 800 may calculate the first pressure value PR1 as the diastolic blood pressure DBP. In addition, the main processor 800 may calculate a pressure value greater than the pressure value corresponding to the peak value PK among the pressure values as a second pressure value PR2. In addition, the main processor 800 may calculate the second pressure value PR2 as the systolic blood pressure SBP.

In a case of the present embodiment, the third pulse wave signal PPG3 fluctuates according to the heartbeat cycle, and may thus reflect a change in blood pressure according to the heartbeat. The display device 1 may accurately calculate blood pressure information of the user based on the third pulse wave signal PPG3.

Also in a case of the present embodiment, the display device calculates an abnormal measurement section and removes and interpolates a pulse wave signal of the abnormal measurement section. In addition, the display device calculates the blood pressure based on the interpolated pulse wave signal, and thus, may accurately measure the blood pressure.

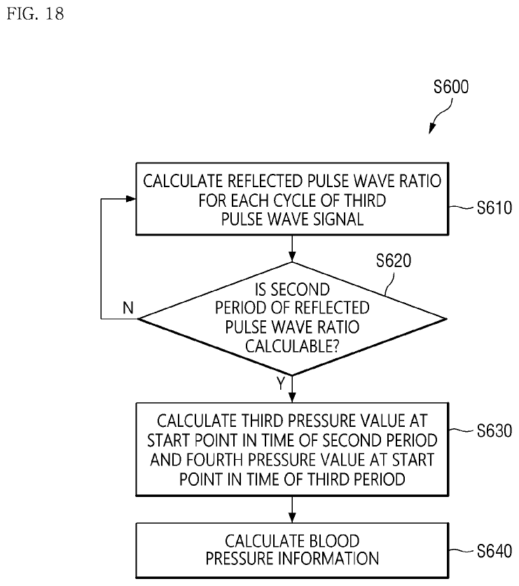

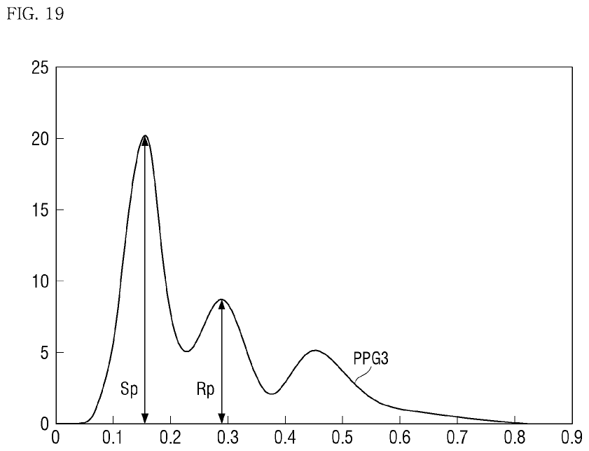

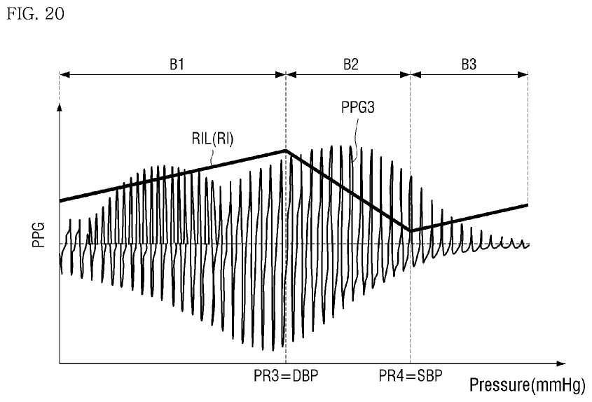

FIG. 18 is a flowchart illustrating a method of calculating a blood pressure according to another embodiment. FIG. 19 is a graph illustrating a waveform of one cycle of a pulse wave signal according to another embodiment. FIG. 20 is a graph illustrating a pulse wave signal and a reflected pulse wave ratio according to another embodiment. Hereinafter, a method in which the display device calculates a blood pressure based on a reflected pulse wave ratio RI will be described with reference to FIGS. 18 to 20.

Referring to FIG. 18, first, a reflected pulse wave ratio RI is calculated for each cycle of the third pulse wave signal PPG3 (S610).

Referring to FIG. 19, in order to calculate the reflected pulse wave ratio RI, the main processor 800 divides a wave cycle of the third pulse wave signal PPG3 according to a period in which a wave according to a heartbeat and a reflected wave of a blood vessel are sequentially generated. According to some embodiments, a cycle of the third pulse wave signal includes a plurality of waveforms, a first waveform and a second waveform of the plurality of waveforms having different amplitudes, and wherein the main processor calculates the reflected pulse wave ratio RI using formula RI=Rp/Sp, wherein Sp is a pulse wave contraction value defined by a peak value of the first waveform, and Rp is a reflected pulse wave value defined by a peak value of the second waveform. For example, one cycle of the third pulse wave signal PPG3 may include a plurality of waveforms having different amplitudes. Accordingly, when a peak value of a waveform having the greatest amplitude among the plurality of waveforms is defined as a pulse wave contraction value, a peak value of a waveform having the second greatest amplitude among the plurality of waveforms is defined as a reflected pulse wave value, the pulse wave contraction value is defined as Sp, the reflected pulse wave value is defined as Rp, and the reflected pulse wave ratio is defined as RI, the reflected pulse wave ratio RI may be calculated by the following Equation (2).

$$RI = \frac{Rp}{Sp} \qquad Equation ( 2 )$$

In summary, the main processor 800 may calculate the peak value of the waveform having the greatest amplitude among the plurality of waveforms of one cycle of the third pulse wave signal PPG3. In addition, the main processor 800 may calculate the peak value of the waveform having the second greatest amplitude among the plurality of waveforms of one cycle of the third pulse wave signal PPG3. In addition, the main processor 800 may calculate the reflected pulse wave ratio RI based on the pulse wave contraction value Sp and the reflected pulse wave value Rp.

Second, the main processor 800 determines whether or not a second period B2 of the reflected pulse wave ratio RI may be calculated (S620). The main processor 800 sequentially stores detection results of reflected pulse wave ratios RI of reflected pulse waves to pulse wave contraction values, and analyzes the stored reflected pulse wave ratios RI. The main processor 800 may continuously convert varies in magnitude of the reflected pulse wave ratios RI into data to analyze a change in magnitude of reflected pulse wave ratio data RIL(RI).

The reflected pulse wave ratio RI includes a first period B1 in which the reflected pulse wave ratio RI fluctuates within a first range, a second period B2 in which the reflected pulse wave ratio RI fluctuates within a second range, and a third period B3 in which the reflected pulse wave ratio RI fluctuates within a third range. For example, the main processor 800 may analyze a reflected pulse wave ratio signal RIL to analyze a first period B1 in which the reflected pulse wave ratio RI is gently changed within a preset range in a saturated state, a second period B2 in which the reflected pulse wave ratio RI is sharply decreased or increased beyond the preset range within a preset period, a third period B3 in which the reflected pulse wave ratio RI is gently changed within the preset range in a saturated state again after it is sharply decreased or increased, and the like.

Here, a width of the first range and a width of the third range may be smaller than a width of the second range. In addition, a gradient of the second period B2 of the reflected pulse wave ratio RI may be greater than a gradient of the first period B1 of the reflected pulse wave ratio RI and a gradient of the third period B3 of the reflected pulse wave ratio RI.

Finally, the main processor 800 calculates a systolic blood pressure SBP, a diastolic blood pressure DBP, and the like, based on the reflected pulse wave ratio RI (S630), and calculates blood pressure information (S640).

According to some embodiments, during a first period the reflected pulse wave ratio fluctuates within a first range, during a second period the reflected pulse wave ratio fluctuates within a second range, and during a third period reflected pulse wave ratio fluctuates within a third range, wherein the first period, the second period, and the third period are consecutive and continuous, and wherein a width of the first range and a width of the third range are smaller than a width of the second range.

According to some embodiments, the main processor 800 may analyze the reflected pulse wave ratio RI to detect a start point in time of the second period B2. In addition, the main processor 800 may calculate a third pressure value PR3 corresponding to the third pulse wave signal PPG3 at the start point in time of the second period B2. In addition, the main processor 800 may calculate the third pressure value PR3 as the diastolic blood pressure DBP. In addition, the main processor 800 may analyze the reflected pulse wave ratio RI to detect a start point in time of the third period B3 after the second period B2. In addition, the main processor 800 may calculate a fourth pressure value PR4 corresponding to the third pulse wave signal PPG3 at the start point in time of the third period B3. In addition, the main processor 800 may calculate the fourth pressure value PR4 as the systolic blood pressure SBP.

In a case of the blood pressure measurement method of the blood pressure measurement system according to the present embodiment, the pressure measurement device may measure the pressure applied to the artery of the user, and the display device 1 may measure the pulse wave signal. The blood pressure measurement system according to the present embodiment may more accurately measure the pressure by applying the pressure to the user's body and measuring the pressure applied to the user's body.

In addition, the display device 1 may calculate the measurement section TT2 in the first pressure signal PSS1 to calculate the second pulse wave signal PPG2 and the second pressure signal PSS2. Accordingly, the display device 1 may calculate the blood pressure of the user based on accurate pressure information and pulse wave information to accurately calculate the blood pressure.

In concluding the detailed description, those skilled in the art will appreciate that many variations and modifications can be made to the embodiments without substantially departing from the principles of the present invention. Therefore, the disclosed embodiments of the invention are used in a generic and descriptive sense only and not for purposes of limitation.

What is claimed is:

1. A blood pressure measurement system comprising:
a display device; and
a pressure measurement device applying a pressure to a user's body according to a pressure control signal and sensing a pressure measurement value,
wherein the display device includes:
a pixel emitting light;
a photo-sensor sensing external light and outputting a first pulse wave signal based on the external light; and
a main processor, wherein the main processor
receives the first pulse wave signal,
outputs the pressure control signal to the pressure measurement device,
receives a first pressure signal including the pressure measurement value from the pressure measurement device, and
calculates a blood pressure based on the first pulse wave signal and the first pressure signal.

2. The blood pressure measurement system of claim 1, wherein the pressure measurement device includes:
a pressure applying part applying the pressure to the user's body;
a pressure sensor sensing an external pressure; and
a controller controlling the pressure applying part.

3. The blood pressure measurement system of claim 2, wherein the main processor controls the pressure applying part so that the pressure measurement value gradually increases during a period of time.

4. The blood pressure measurement system of claim 2, wherein the pressure measurement device further includes a wireless communication module receiving the pressure control signal.

5. The blood pressure measurement system of claim 2, wherein the controller controls the pressure sensor and the photo-sensor to be synchronized and to simultaneously sense the external pressure from the user's body and light reflected from the user's body, respectively.

6. The blood pressure measurement system of claim 1, wherein the main processor calculates, based on the first pressure signal, a non-measurement section and a measurement section for a period of time, wherein during the non-measurement section the pressure measurement value is constant and during the measurement section the pressure measurement value varies.

7. The blood pressure measurement system of claim 6, wherein the main processor, during a section corresponding to the measurement section, extracts the first pressure signal to calculate a second pressure signal and extracts the first pulse wave signal to calculate a second pulse wave signal.

8. The blood pressure measurement system of claim 7, wherein the main processor further calculates a third pulse wave signal including a pulse wave signal value based on the second pulse wave signal and the second pressure signal.

9. The blood pressure measurement system of claim 8, wherein the main processor generates a peak detection signal based on an amplitude corresponding to a peak of a cycle of the third pulse wave signal.

10. The blood pressure measurement system of claim 9, wherein the main processor calculates a peak value of the peak detection signal, a pressure value corresponding to the peak value of the peak detection signal, a diastolic blood pressure, a systolic blood pressure, and a mean blood pressure, wherein the diastolic blood pressure is lower than the pressure value corresponding to the peak value and the systolic blood pressure is higher than the pressure value corresponding to the peak value.

11. The blood pressure measurement system of claim 10, wherein the main processor calculates the mean blood pressure as the pressure value corresponding to the peak value.

12. The blood pressure measurement system of claim 10, wherein the diastolic blood pressure is smaller than a pressure value corresponding to 60% to 80% of the peak value and the systolic blood pressure is greater than the pressure value corresponding to 60% to 80% of the peak value.

13. The blood pressure measurement system of claim 8, wherein a cycle of the third pulse wave signal includes a plurality of waveforms, a first waveform and a second waveform of the plurality of waveforms having different amplitudes, and wherein the main processor calculates reflected pulse wave ratio RI using formula RI=Rp/Sp, wherein Sp is a pulse wave contraction value defined by a peak value of the first waveform, and Rp is a reflected pulse wave value defined by a peak value of the second waveform.

14. The blood pressure measurement system of claim 13, wherein during a first period the reflected pulse wave ratio fluctuates within a first range, during a second period the reflected pulse wave ratio fluctuates within a second range, and during a third period reflected pulse wave ratio fluctuates within a third range, wherein the first period, the second period, and the third period are consecutive and continuous, and wherein a width of the first range and a width of the third range are smaller than a width of the second range.

15. The blood pressure measurement system of claim 14, wherein the main processor
detects a start point of the second period based on the reflected pulse wave ratio,
calculates a third pressure value corresponding to the first pulse wave signal at the start point of the second period,
sets the third pressure value as the diastolic blood pressure,
calculates a fourth pressure value corresponding to the first pulse wave signal at a start point of the third period after the second period,
and sets the fourth pressure value as the systolic blood pressure.

16. A blood pressure measurement method of using a blood pressure measurement system including a pressure measurement device applying a pressure to a user's body and sensing a pressure measurement value; and a display device including a pixel emitting light and a photo-sensor sensing external light and outputting a first pulse wave signal based on the external light, the blood pressure measurement method comprising:
applying a pressure control signal to the pressure measurement device for controlling the pressure measurement device;
receiving a first pressure signal including the pressure measurement value during a period of time from the pressure measurement device;
receiving the first pulse wave signal from the photo-sensor;
calculating, based on the first pressure signal, a non-measurement section and a measurement section for a period of time, wherein during the non-measurement section the pressure measurement value is constant and during the measurement section the pressure measurement value varies;
extracting the first pressure signal in a section corresponding to the measurement section to calculate a second pressure signal and extracting the first pulse wave signal in the section corresponding to the measurement section to calculate a second pulse wave signal; and
calculating a blood pressure based on the second pulse wave signal and the second pressure signal.

17. The blood pressure measurement method of claim 16, wherein the first pressure signal gradually increases in the measurement section.

18. The blood pressure measurement method of claim 16, further comprising calculating a third pulse wave signal including a pulse wave signal value based on the second pulse wave signal and the second pressure signal and generating a peak detection signal based on an amplitude corresponding to a peak of a cycle of the third pulse wave signal.

19. The blood pressure measurement method of claim 18, further comprising calculating a peak value of the peak detection signal and a pressure value corresponding to the peak value of the peak detection signal and calculating a diastolic blood pressure lower than the pressure value, a systolic blood pressure higher than the pressure value, and a mean blood pressure.

20. The blood pressure measurement method of claim 19, wherein the mean blood pressure is calculated as the pressure value corresponding to the peak value.
